(19) United States
(12) Patent Application Publication
(10) Pub. No.: US 2024/0009421 A1
(43) Pub. Date: Jan. 11, 2024

(54) EMOTIONAL CARE APPARATUS AND METHOD THEREOF

(71) Applicants: Hyundai Motor Company, Seoul (KR); Kia Corporation, Seoul (KR); IUCF-HYU (Industry-University Cooperation Foundation Hanyang University), Seoul (KR)

(72) Inventors: Ki Chang Kim, suwon (KR); Tae kun Yun, Anyang (KR); Eun Soo Jo, Hwaseong (KR); Dong Chul Park, Anyang (KR); Eun Ju Jeong, Seoul (KR); Ji Yeon Shin, Seoul (KR)

(21) Appl. No.: 17/981,400

(22) Filed: Nov. 5, 2022

(30) Foreign Application Priority Data
Jul. 5, 2022 (KR) 10-2022-0082545

(51) Int. Cl. A61M 21/02; H04R 1/02; H04R 1/40; H04R 3/12; H04R 1/26; G06F 3/16; G08B 7/00

(52) U.S. Cl. CPC A61M 21/02; H04R 1/025; H04R 1/403; H04R 3/12; H04R 1/26; G06F 3/165; G08B 7/00; H04R 2499/13; H04R 2430/01; A61M 2021/0027

(57) ABSTRACT

An emotional care apparatus based on a sound and a method thereof are provided. The emotional care apparatus includes a sound output device that outputs a sound to speakers and a processor electrically connected with the sound output device. The processor selects an emotional care mode, separates a sound source for each instrument from music content based on the emotional care mode, distributes the sound source for each instrument to the speakers, and controls the sound output device to play and output the sound source for each instrument to the distributed speakers.

### CROSS-REFERENCE TO RELATED APPLICATION

This application claims under 35 U.S.C. § 119(a) the benefit of and priority to Korean Patent Application No. 10-2022-0082545, filed in the Korean Intellectual Property Office on Jul. 5, 2022, the entire contents of which are incorporated herein by reference.

### BACKGROUND

### Technical Field

The present disclosure relates to an emotional care apparatus based on a sound and a method thereof.

### Background

Modern people are exposed to a lot of stress in their daily life. When such stress is accumulated, it may appear as a psychological or physiological symptom in the body. Thus, there has been a growing interest in a management method for suitably managing stress in daily life. Therefore, there has been an increase in demand for content capable of helping people manage their stress.

### SUMMARY

The present disclosure has been made to solve the above-mentioned problems occurring in the related art while advantages achieved by the related art are maintained intact.

An aspect of the present disclosure provides an emotional care apparatus for separating a sound source for each instrument based on passenger emotion when playing music content in a vehicle and distributing the separated sound source for each instrument to a speaker to play the sound source for each instrument and a method thereof.

Another aspect of the present disclosure provides an emotional care apparatus for exciting a vibration seat based on the sound source for each instrument, which is separated from, music content which is being played in a vehicle and a method thereof.

The technical problems addressed by the present disclosure are not limited to the aforementioned problems, and any other technical problems not mentioned herein will be clearly understood from the following description by those skilled in the art to which the present disclosure pertains.

According to an aspect of the present disclosure, an emotional care apparatus may include a sound output device that is configured to selectively output a sound to a plurality of speakers and a processor in communication with the sound output device. The processor may select an emotional care mode, may separate a sound source comprising a plurality of instrument sounds from music content based on the emotional care mode, may distribute the sound source and separated instrument sounds to at least one distributed speaker of the plurality of speakers, and may control the sound output device to play and output the sound source and separated instrument sounds to the at least one distributed speaker. The processor may distribute the sound source and separated instrument sounds to the plurality of speakers based on a position of a passenger in a vehicle.

The processor may distribute the sound source and separated instrument sounds to the plurality of speakers based on a frequency for each instrument sound of the plurality of instrument sounds.

The processor may correct a volume level of the sound source and separated instrument sounds depending on the emotional care mode.

The processor may modulate a waveform of the sound source and separated instrument sounds depending on the emotional care mode.

The processor may generate an emotional vibration signal based on the sound source for each instrument sound of the plurality of instrument sounds and may control a vibration seat to be excited based on the emotional vibration signal.

The processor may convert the sound source and separated instrument sounds into a converted vibration signal, may synthesize a modulation signal of a main vibration signal and a sub-vibration signal with the converted vibration signal into a synthesized vibration signal, and may correct the synthesized vibration signal to generate the emotional vibration signal.

The main vibration signal may be a sine wave. The sub-vibration signal may be a square wave, a triangle wave, or a sawtooth wave.

The processor may generate a first vibration at a first point of a seat back based on the main vibration signal and may generate a second vibration at a second point adjacent the seat back and a seat cushion based on the sub-vibration signal.

The processor may determine the emotional care mode based on at least one of: a user input, a vehicle environment, and/or a passenger emotion state received from a user interface.

According to another aspect of the present disclosure, an emotional care method may include selecting, by a processor, an emotional care mode, separating, by the processor, a sound source including a plurality of instrument sounds into separated instrument sounds from music content based on the emotional care mode, distributing, by the processor, the sound source and separated instrument sounds to at least one distributed speaker of a plurality of speakers in a vehicle, and controlling, by the processor, a sound output device to output the sound source and separated instrument sounds to the at least one distributed speaker.

The distributing of the sound source and separated instrument sounds to the plurality of speakers in the vehicle may include distributing the sound source for and separated instrument sounds to the plurality of speakers based on a position of a passenger in the vehicle.

The distributing of the sound source and separated instrument sounds to the plurality of speakers in the vehicle may include distributing the sound source for each instrument sound of the plurality of instrument sounds to the speakers based on a frequency for each instrument sound of the plurality of instrument sounds.

The distributing of the sound source and separated instrument sounds to the plurality of speakers in the vehicle may include correcting a volume level of the sound source and separated instrument sounds depending on the emotional care mode.

The distributing of the sound source and separated instrument sounds to the plurality of speakers in the vehicle may include modulating a waveform of the sound source and separated instrument sounds depending on the emotional care mode.

The emotional care method may further include generating an emotional vibration signal based on the sound source for each instrument sound of the plurality of instrument sounds and controlling a vibration seat to be excited based on the emotional vibration signal.

The generating of the emotional vibration signal may include converting the sound source and separated instrument sounds into a converted vibration signal, synthesizing a modulation signal of a main vibration signal and a sub-vibration signal with the converted vibration signal into a synthesized vibration signal, and correcting the synthesized vibration signal to generate the emotional vibration signal.

The controlling of the vibration seat to be excited may include generating a first vibration at a first point of a seat back based on the main vibration signal and generating a second vibration at a second point adjacent the seat back and a seat cushion based on the sub-vibration signal.

The selecting of the emotional care mode may include determining the emotional care mode based on at least one of: a user input, a vehicle environment, and/or a passenger emotion state received from a user interface.

### BRIEF DESCRIPTION OF THE DRAWINGS

The above and other objects, features and advantages of the present disclosure will be more apparent from the following detailed description taken in conjunction with the accompanying drawings:

FIG. 1 is a block diagram illustrating a configuration of an emotional care apparatus according to embodiments of the present disclosure;

FIG. 2 is a drawing illustrating an example of arranging speakers according to embodiments of the present disclosure;

FIG. 3 is a drawing illustrating an example of installing vibration generators in a vehicle seat according to embodiments of the present disclosure;

FIG. 4 is a drawing for describing a per-speaker sound source distribution algorithm according to embodiments of the present disclosure;

FIG. 5 is a drawing illustrating a configuration of a virtual environment sound tuning simulator according to embodiments of the present disclosure;

FIG. 6 is a drawing illustrating an example of forming a sound zone according to a passenger position according to embodiments of the present disclosure;

FIG. 7 is a drawing illustrating another example of forming a sound zone according to a passenger position according to embodiments of the present disclosure;

FIG. 8 is a flowchart illustrating an emotional care method according to embodiments of the present disclosure;

FIG. 9 is a drawing for describing a sound-based vibration classification algorithm according to embodiments of the present disclosure;

FIG. 10 is a flowchart illustrating a method for implementing an emotional vibration according to embodiments of the present disclosure; and

FIG. 11 is a flowchart illustrating a method for determining a vibration pattern and a vibration exciting force according to embodiments of the present disclosure.

### DETAILED DESCRIPTION

Hereinafter, some embodiments of the present disclosure will be described in detail with reference to the exemplary drawings. In the drawings, the same reference numerals will be used throughout to designate the same or equivalent elements. In addition, a detailed description of well-known features or functions will be ruled out in order not to unnecessarily obscure the gist of the present disclosure.

In describing the components of the embodiment according to the present disclosure, terms such as first, second, “A”, “B”, (a), (b), and the like may be used. These terms are only used to distinguish one element from another element, but do not limit the corresponding elements irrespective of the order or priority of the corresponding elements. Furthermore, unless otherwise defined, all terms including technical and scientific terms used herein are to be interpreted as is customary in the art to which the present disclosure belongs. Such terms as those defined in a generally used dictionary are to be interpreted as having meanings equal to the contextual meanings in the relevant field of art, and are not to be interpreted as having ideal or excessively formal meanings unless clearly defined as having such in the Present application.

Embodiments of the present disclosure may separate a sound source for each instrument from music content when playing the music content based on driver emotion modeling in a vehicle, may distribute the separated sound source for each instrument to a speaker to play the sound source for each instrument, and may control a vibration seat to be excited based on the separated sound source for each instrument to help vehicle passengers refresh themselves.

Embodiments of the present disclosure may provide an emotional care solution based on three concepts. To this end, embodiments of the present disclosure establishes a driver emotion category using a three-dimensional (3D) emotion analysis, that is, an emotion analysis of pleasure, arousal, and dominance dimensions. The driver emotion category may be classified as a safe persona, a fun persona, or a healthy persona.

Furthermore, to generate excitation in a vibration seat based on a sound source for each instrument depending on the emotional care solution (or driver emotion modeling or a mood), embodiments of the present disclosure may derive multisensory stimulation of sound-based vibration by means of a study on a sound-based vibration and haptic correlation and may make up a musical instrument by means of musical emotion definition based on driver emotion modeling and may derive a keyword for each situation.

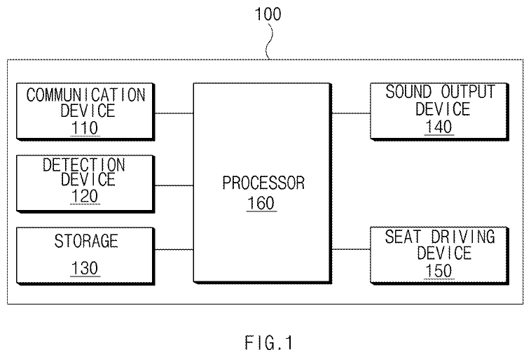

FIG. 1 is a block diagram illustrating a configuration of an emotional care apparatus according to embodiments of the present disclosure.

Referring to FIG. 1, an emotional care apparatus 100 may include a communication device 110, a detection device 120, a storage 130, a sound output device 140, a seat driving device 150, and a processor 160.

The communication device 110 may assist the emotional care apparatus 100 to perform wired communication (e.g., an Ethernet, a local area network (LAN), a controller area network (CAN), or the like) and/or a wireless communication (e.g., wireless-fidelity (Wi-Fi), Bluetooth, long term evolution (LIE), or the like) with an electronic device (e.g., a smartphone, an electronic control unit (ECU), a tablet, a personal computer, or the like) which is located inside and/or outside a vehicle. The communication device 110 may include a transceiver which transmits and/or receives a signal (or data) using at least one antenna.

The detection device 120 may detect vehicle information (e.g., driving information and/or vehicle environment information), driver information, passenger information, and/or the like. The detection device 120 may detect vehicle information, such as a vehicle speed, seat information, a motor revolution per minute (RPM), an accelerator pedal opening amount, a throttle opening amount, a vehicle interior temperature, and/or a vehicle exterior temperature, using at least one sensor and/or at least one ECU, which are/is mounted on the vehicle. An accelerator position sensor (APS), a throttle position sensor, a global positioning system (GPS) sensor, a wheel speed sensor, a temperature sensor, a microphone, an image sensor, an advanced driver assistance system (ADAS) sensor, a 3-axis accelerometer, an inertial measurement unit (IMU), and/or the like may be used as the at least one sensor. The at least one ECU may be a motor control unit (MCU), a vehicle control unit (VCU), and/or the like. The detection device 120 may detect driver information and passenger information using a pressure sensor, an ultrasonic sensor, a radar, an image sensor, a microphone, a driver monitoring system (DMS), and/or the like.

The storage 130 may store a sound source distribution algorithm for each speaker, a sound-based vibration classification algorithm, and/or the like. The storage 130 may store a sound (or a sound source) such as a music sound (or music content), a virtual sound, and/or a driving sound. The music content may be created according to a guideline on musical features (e.g., a musical structure, musical representation, a tone, and the like) based on driver emotion modeling (or a service concept) and a guideline on features for each persona.

A pre-training database by an artificial intelligence-based emotional vibration algorithm may be constructed in the storage 130. It may be identified whether a sound source sample for each instrument is similar to an emotion to be induced in a concept stage by constructing the pre-training database by the artificial intelligence-based emotional vibration algorithm. The construction of the pre-training database may be accomplished by the following procedure. First of all, the processor 160 may play a sound source for each instrument based on driver emotion modeling and may classify a sound source for generating a vibration based on a waveform of the played sound source. In other words, the processor 160 may analyze a track for each sound source on the basis of a sound source playback time, an instrument group, pitch, or the like to determine whether the sound source includes a track for generating a vibration. Correlation equation t=(60/BPM)*5(number of beats), where PPM is beats per minute. Next, the processor 160 may map a sound source track classified as the sound source for generating the vibration to a vibration actuator (i.e., a vibration generator). The processor 160 may analyze the first 4 bars of the verse part presented after the intro to analyze a mode correlation for each mood. The processor 160 may perform preprocessing (i.e., filtering) of maintaining a waveform of a sound source for each track and removing an unnecessary frequency band using a recursive linear filter. The processor 160 may synthesize the preprocessed sound source for each track with a waveform for each emotional care mode. In other words, the processor 160 may synthesize a sine wave with the preprocessed sound source for each track, when the emotional care mode is a meditation mode, may synthesize a triangle wave with the preprocessed sound source for each track, when the emotional care mode is a stress relief mode, may synthesize a square wave with the preprocessed sound source for each track, when the emotional care mode is a healing mode to generate an emotional vibration. Thereafter, the processor 160 may set a regression model design and a hypothesis and may analyze audio data. The processor 160 may generate experimental tools, for example, a questionnaire, an experimental method, detailed settings, and the like and may construct a pre-training database by establishing an experimental design.

The storage 130 may be a non-transitory storage medium which stores instructions executed by the processor 160. The storage 130 may include at least one of storage media such as a random access memory (RAM), a static RAM (SRAM), a read only memory (ROM), a programmable ROM (PROM), an electrically erasable and programmable ROM (EEPROM), an erasable and programmable ROM (EPROM), a hard disk drive (HDD), a solid state disk (SSD), an embedded multimedia card (eMMC), universal flash storage (UPS), or web storage.

The sound output device 140 may play and output a sound source which is previously stored or is streamed in real time to the outside. The sound output device 140 may include an amplifier, speakers (e.g., a twitter, a woofer, a subwoofer, and the like), and/or the like. The amplifier may amplify an electrical signal of a sound played from the sound output device 140. A plurality of speakers may be installed at different positions inside and/or outside the vehicle. The speaker may convert the electrical signal amplified by the amplifier into a sound wave.

The sound output device 140 may play and output music content, a sound source for each instrument, a virtual sound, and/or a healing sound to the interior and exterior of the vehicle under an instruction of the processor 160. The sound output device 140 may include a digital signal processor (DSP), microprocessors, and/or the like. The sound output device 140 may output music content, a sound source for each instrument, a virtual sound, and/or a healing sound to speakers (e.g., a 3way speaker and a Sway speaker) loaded into the vehicle. Furthermore, the sound output device 140 may output a virtual sound and/or a healing sound to speakers (or external amplifiers) mounted on the exterior of the vehicle.

The seat driving device 150 may control at least one vibration generator mounted on a vehicle seat to generate a vibration (or a vibration signal). The seat driving device 150 may adjust a vibration pattern, vibration intensity, a vibration frequency, and/or the like. At least one vibration generator may be installed in a specific position of the vehicle seat, for example, a seat back, a seat cushion, a leg rest, and/or the like. The vibration generator may control at least one vibration to perform vehicle seat excitation.

The processor 160 may be electrically connected with the respective components 110 to 150. The processor 160 may control operations of the respective components 110 to 150. The processor 160 may include at least one of processing devices such as an application specific integrated circuit (ASIC), a digital signal processor (DSP), programmable logic devices (PLD), field programmable gate arrays (FPGAs), a central processing unit (CPU), microcontrollers, or microprocessors.

The processor 160 may determine driver emotion modeling, that is, an emotional care mode or mood or the like based on a user input transmitted from a user interface. The processor 160 may determine driver emotion modeling with regard to a vehicle environment and/or an emotional state of a passenger.

The processor 160 may play music content based on the driver emotion modeling. The processor 160 may separate a sound source for each instrument from the played music content. At this time, the processor 160 may separate the sound source for each instrument from music content based on instrument composition matched with the driver emotion modeling. An instrument used when music content which is emotional content is designed may be a piano, a chromatic percussion, a guitar, a bass, strings (solo, ensemble), winds (brass, reed, pipe), synth effects (Fx), a percussion (e.g., a drum), a pad, or the like. The instrument composition based on the driver emotion modeling is as follows.

Stress relief: Piano, Guitar, Bass, Strings, Winds, Percussion

Meditation: Piano, Percussion, Pad

Healing: Chromatic Percussion, Guitar, Synth Effects, Percussion, Pad

The processor 160 may distribute the separated sound source for each instrument to speakers. The processor 160 may distribute the sound source for each instrument to the speakers using a per-speaker sound source distribution algorithm. In other words, the processor 160 may distribute the sound source for each instrument based on the driver emotion modeling. According to an embodiment, because of separating a sound source for each instrument based on a sound characteristic for each frequency and the driver emotion modeling and playing the sound source for each instrument using a speaker for each mood, the processor 160 may facilitate emotional care with the feeling of an orchestra.

The processor 160 may control a sound image depending on a position of a passenger in the vehicle. As an example, when there is a passenger (i.e., a driver) on only the driver's seat in the vehicle, the processor 160 may control a phase image to be located in a central portion of the front of the vehicle. As another example, when there are passengers on the driver's seat and the rear VIP seat in the vehicle, the processor 160 may adjust a location of the phase image such that the sound is widely distributed to the center of the rear of the center console. As such, embodiments of the present disclosure may prevent a phenomenon in which the sound image is skewed due to a binaural effect, a Haas effect, and the like by means of sound image control.

The processor 160 may generate a vibration based on the sound source for each instrument according to the emotional care mode. The processor 160 may generate a main vibration signal based on the sound source for each instrument according to the emotional care mode. The processor 160 may modulate a waveform of a sub-vibration signal according to the emotional care mode. The processor 160 may synthesize the main vibration signal with the modulated sub-vibration signal. The processor 160 may control the seat driving device 150 to excite a seat back based on the main vibration signal and excite a seat waist and thigh based on the sub-vibration signal. For example, when the emotional care mode is the meditation mode, the processor 160 may generate a main vibration of piano emotion in a seat back location and may generate a sub-vibration in a seat waist and thigh location.

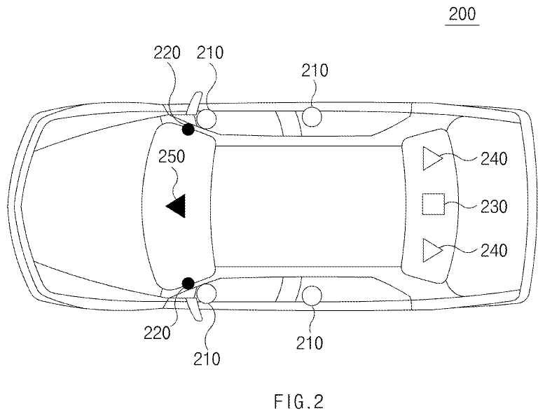

FIG. 2 is a drawing illustrating an example of arranging speakers according to embodiments of the present disclosure.

Referring to FIG. 2, a speaker system 200 may be applied to the interior of a vehicle and may be implemented as a Sway system. The speaker system 200 may include woofers 210, tweeters 220, a subwoofer 230, first mid-range speakers 240, and a second mid-range speaker 250. Each of the woofers 210 may be a speaker for a high frequency (100 Hz to 300 Hz). Each of the tweeters 220 may be a speaker for a mid-frequency (3 KHz to 20 KHz). The subwoofer 230 may be a speaker for a low frequency (20 Hz to 100 Hz). The first mid-range speakers 240 may be installed at both sides in the rear of the vehicle. Each of the first mid-range speakers 240 may be a speaker for a mid-frequency (300 Hz to 3 KHz). The second mid-range speaker 250 may be installed in the front center of the vehicle and may be a speaker for a mid-frequency.

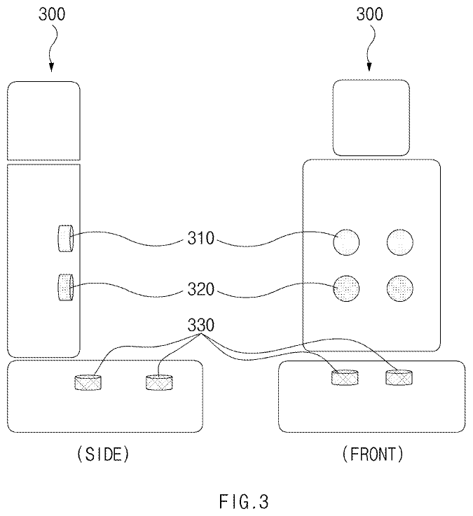

FIG. 3 is a drawing illustrating an example of installing vibration generators in a vehicle seat according to embodiments of the present disclosure.

Referring to FIG. 3, first to third vibration generators 310 to 330 may be installed at predetermined locations in a vehicle seat 300. Each of the first to third vibration generators 310 to 330 may be implemented as a tactile transducer (TTD). The first vibration generator 310, the second vibration generator 320, and the third vibration generator 330 may be installed at different locations of the vehicle seat 300. The first vibration generator 310 may generate a high-frequency vibration. The second vibration generator 320 may generate a mid-frequency vibration. The third vibration generator 330 may generate a low-frequency vibration.

The first vibration generator 310 may excite a vibration in the vehicle seat 300 based on a main vibration signal. The second vibration generator 320 and the third vibration generator 330 may excite a vibration in the vehicle seat 300 based on a sub-vibration signal or a modulated sub-vibration signal. For example, the first vibration generator 310 may generate a vibration of a sine wave corresponding to a melody of music content, the second vibration generator 320 may generate a vibration of a square wave or a sine wave corresponding to a harmony of the music content, and the third vibration generator 330 may generate a vibration of a triangle wave or a sawtooth wave corresponding to bass of the music content.

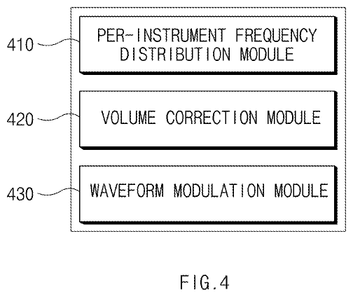

FIG. 4 is a drawing for describing a per-speaker sound source distribution algorithm according to embodiments of the present disclosure.

A processor 160 of FIG. 1 may implement realism through distance perception, that is, a chamber orchestra effect, based on the per-speaker sound source distribution algorithm. The per-speaker sound source distribution algorithm may include a per-instrument frequency distribution module 410, a volume correction module 420, and a waveform modulation module 430.

The per-instrument frequency distribution module 410 may distribute a frequency for each instrument depending on an emotional care mode. In other words, the per-instrument frequency distribution module 410 may distribute a frequency based on a frequency of a sound source for each instrument (or a pitch of a sound).

The volume correction module 420 may correct the volume of the sound source for each instrument depending on the emotional care mode to induce an emotional change.

The waveform modulation module 430 may change a difference according to a tone of the sound source for each instrument, that is, a temporal change in waveform. In other words, the waveform modulation module 430 may change an amplitude and a period of the sound source for each instrument.

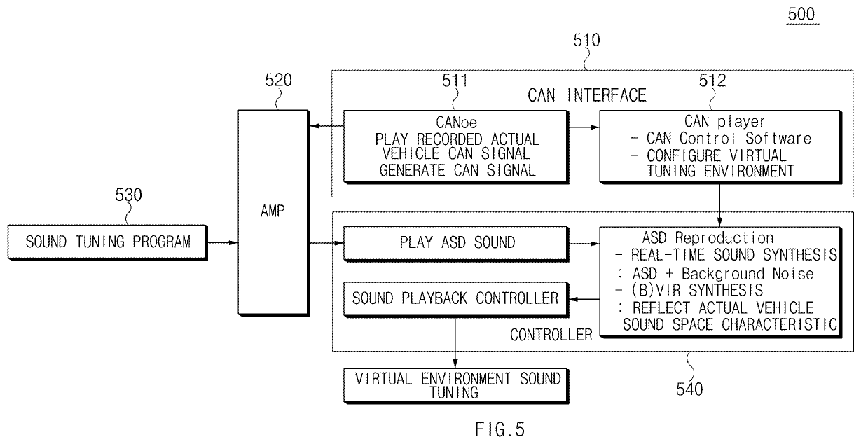

FIG. 5 is a drawing illustrating a configuration of a virtual environment sound tuning simulator according to embodiments of the present disclosure.

A virtual environment sound tuning simulator 500 may perform virtual environment sound turning using ASD hardware in loop simulation (HiLS). The virtual environment sound tuning simulator 500 may include a CAN interface 510, an AMP 520, a sound tuning program 530, and a controller 540.

The CAN interface 510 may record, play, generate, or transmit and receive actual vehicle driving information between the respective devices. In other words, the CAN interface 510 may serve as a CAN signal transceiver which transmits and receives a CAN signal collected in an actual vehicle with the AMP 520 and the controller 540. The CAN interface 510 may generate a CAN signal including a parameter calculated by the virtual environment sound tuning simulator 500 and may transmit the generated CAN signal to the AMP 520.

The CAN interface 510 may include a controller area network open environment (CANoe) 511 and a CAN player 512, which may play the same signal as the vehicle or may manipulate the obtained signal using a CAN signal obtained in the vehicle and may transmit and receive a CAN signal between the AMP 520 and the controller 540.

The AMP 520 may receive a tuning parameter of the sound tuning program 530. The AMP 520 may calculate an output value according to the turning parameter and the CAN signal.

The controller 540 may perform, the overall operation of the virtual environment sound tuning simulator 500, may store and manage default interior sound data generated by recording a noise, vibration, harshness (NVH) sound of the actual vehicle, may store and manage sound field characteristic information (e.g., a binaural vehicle impulse response (BVIR)) from a sound source (e.g., a speaker) in the actual vehicle to ears of a person, and may generate, collect, and process a CAN signal capable of identifying an operation state of the vehicle.

The controller 540 may play an ASD sound based on the output value (or an output signal) calculated by the AMP 520. The controller 540 may synthesize a sound (e.g., background noise) recorded in the actual vehicle with the played ASD sound to generate a composite sound. Furthermore, the controller 540 may reflect an actual vehicle sound space characteristic, that is, BVIR information in the generated composite sound to generate a final composite sound.

The controller 540 may include a sound playback controller. The sound playback controller may output the final composite sound. In other words, the sound playback controller may perform sound tuning of the final composite sound in a virtual environment.

The controller 540 may allow a user to listen to the tuned sound using a VR simulator which simulates a virtual driving environment and may perform a verification procedure by means of hearing experience feedback on the tuned sound. The controller 540 may repeatedly perform verification of the tuned sound and sound tuning based on the verified result to provide hearing experience of an actual vehicle level.

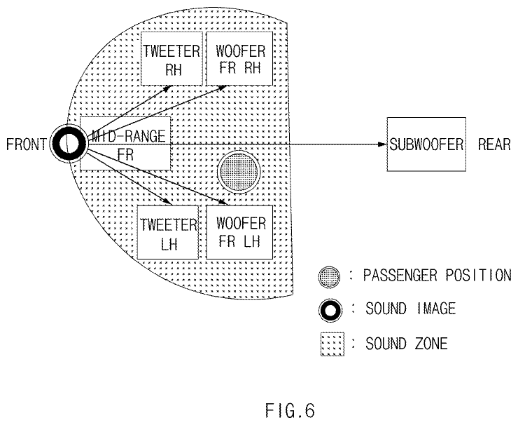

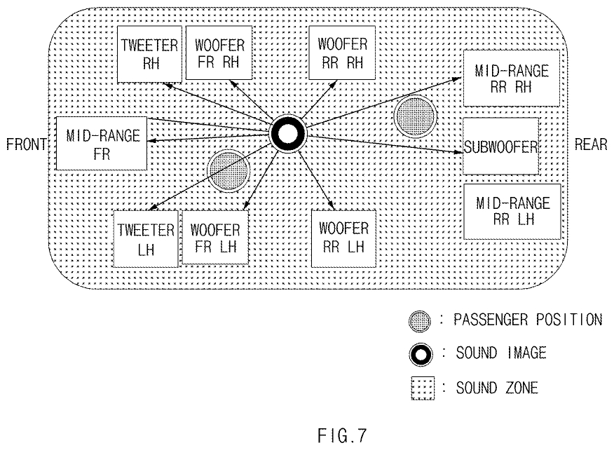

FIG. 6 is a drawing illustrating an example of forming a sound zone according to a passenger position according to embodiments of the present disclosure. FIG. 7 is a drawing illustrating another example of forming a sound zone according to a passenger position according to embodiments of the present disclosure.

Referring to FIG. 6, when only a driver rides in a vehicle, a processor 160 of FIG. 1 may control a sound image to be located in a front center of the vehicle. As the sound image is located in the front center, a sound zone may also be formed in the front of the vehicle.

Referring to FIG. 7, when passengers exist in the driver's seat and the rear VIP seat in the vehicle, the processor 160 may move a sound image from a front center of the vehicle to the rear of the center console of the vehicle. As the sound image moves to the rear of the center console, a sound zone may be formed in the entire area in the vehicle.

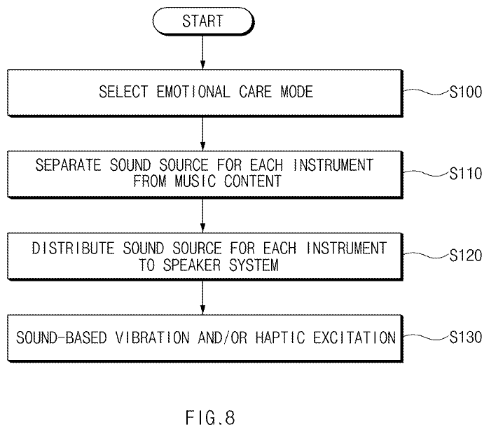

FIG. 8 is a flowchart illustrating an emotional care method according to embodiments of the present disclosure.

Referring to FIG. 8, in S110, a processor 160 of FIG. 1 may select an emotional care mode. The emotional care mode may be an emotional care solution based on driver emotion modeling, which may be divided into a stress relief mode (or safe driving), a meditation mode (or healthy driving), and a healing mode (or fun driving). The processor 160 may select the emotional care mode based on at least one of a user input, a driving environment, or a passenger state.

In S110, the processor 160 may distribute music content based on the emotional care solution played in the vehicle as a sound source for each instrument. In other words, the processor 160 may extract (or separate) a sound source for each instrument from the music content.

In S120, the processor 160 may distribute the sound source for each instrument to a speaker system. The processor 160 may distribute the sound source for each instrument to the speaker system using a per-speaker sound source distribution algorithm. At this time, the per-speaker sound source distribution algorithm may distribute a frequency for each instrument (or a pitch of a sound) based on driver emotion modeling (i.e., an emotional care mode), may induce an emotional change by correcting volume (or intensity of the sound), and may implement a chamber orchestra effect (i.e., realism through distance perception) due to a difference in a temporal change of a waveform (or a tone of the sound).

In S130, the processor 160 may perform sound-based vibration and/or haptic excitation. The processor 160 may select a triangle wave as a main vibration, when the emotional care mode is a stress relief mode, may select a sine wave as the main vibration, when the emotional care mode is a meditation mode, and may select a square wave as the main vibration, when the emotional care mode is a healing mode. A sawtooth wave may be used as a sub-vibration. The sub-vibration may be assigned to improve the emptiness of an interval except for the main vibration. The processor 160 may modulate the sub-vibration using pulse amplitude modulation (PAM), pulse width modulation (PM), and/or pule position modulation (PPM). The processor 160 may modulate a pulse amplitude, a pulse width, and a pulse position of an original waveform of the sub-vibration with regard to a difference in temporal change in sound source waveform for each instrument.

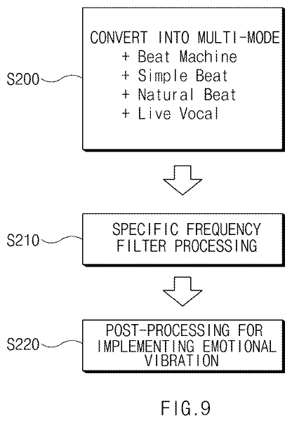

FIG. 9 is a drawing for describing a sound-based vibration classification algorithm according to embodiments of the present disclosure.

In S200, a processor 160 of FIG. 1 may convert a sound source for each instrument into a vibration signal of a multi-mode. The multi-mode may be divided into four types, that is, a beat machine, a simple beat, a natural beat, and a live vocal. The beat machine may be applied to K-pop, hip-hop, or the like. The simple beat may be applied to all music. The natural beat may be applied to classic music. The live vocal may be applied to blues, jazz, or the like.

In S210, the processor 160 may perform specific frequency filter processing for the converted vibration signal. The processor 160 may perform filtering to prevent a sense of difference due to high-pitched excessive vibration excitation. The processor 160 may differently assign a vibration to a seat back (or an upper end) and a seat cushion (or a lower end) for emotional vibration for each instrument using a low-pitched filter.

In S220, the processor 160 may perform post-processing for implementing emotional vibration for the filter-processed vibration signal. Because of adjusting the amount of vibration using an attack, delay, release, sustain (ADSR) curve, the processor 160 may more emotionally deliver the vibration. The processor 160 may determine how to generate, reduce, draw, and remove the vibration, when receiving the vibration signal. A compressor and a limiter may limit an input, when excessively receiving a load. When a signal over a certain signal is excessively received, the compressor may reduce and generate the signal at a certain rate. When a signal over an input signal provided by hardware is received or when a vibration which may harm the human body occurs, the limiter may limit that a vibration over a certain signal does not occur. A gate and an expansor may assign a small vibration to an empty interval. The gate has a scheme which generates a signal when the signal over the certain signal is generated while not generating a vibration signal when the vibration signal is insignificantly small. The expansor has a scheme which enlarges the signal over the certain signal to generate the amount of vibration which is input in advance, when the signal over the certain signal is received.

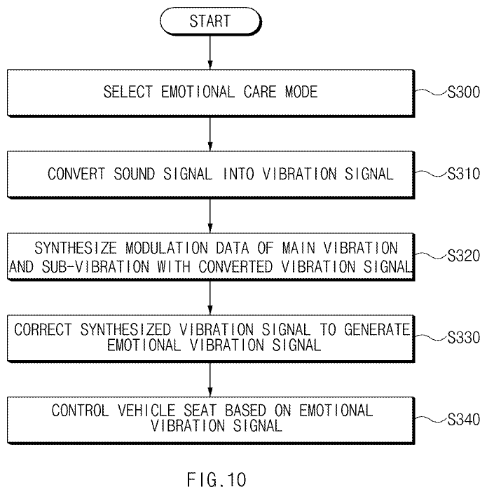

FIG. 10 is a flowchart illustrating a method for implementing an emotional vibration according to embodiments of the present disclosure.

In S300, a processor 160 of FIG. 1 may select an emotional care mode. The processor 160 may determine the emotional care mode depending on a user input. The processor 160 may determine the emotional care mode based on a vehicle environment and/or an emotional state of a passenger. The processor 160 may select the emotional care mode based on a pre-training database by an artificial intelligence-based emotional vibration algorithm. The emotional care mode may be divided into a meditation mode, a stress relief mode, and a healing mode.

In S310, the processor 160 may convert a sound signal into a vibration signal. The processor 160 may implement a vibration multi-mode based on a sound. The vibration multi-mode may include a beat machine, a simple beat, a natural beat, a live vocal, and the like.

In S320, the processor 160 may synthesize modulation data of a main vibration and a sub-vibration with the modulated vibration signal. The main vibration may be a sine wave, and the sub-vibration may be a square wave, a triangle wave, and/or a sawtooth wave. The processor 160 may perform modulation using a modulation scheme of at least one of a pulse amplitude, a pulse width, or a pulse position of the main vibration and the sub-vibration.

In S330, the processor 160 may correct the synthesized vibration signal to generate an emotional vibration signal. The processor 160 may determine a frequency value suitable for a back and thighs in the synthesized vibration signal. The processor 160 may determine a level, a time, or an optimal pattern value of an individual actuator based on the synthesized vibration signal. The processor 160 may correct a vibration exciting force according to a sitting posture or a driving sound pattern.

In S340, the processor 160 may control a vehicle seat based on the emotional vibration signal. The processor 160 may control a seat driving device 150 of FIG. 1 to excite a vibration in the vehicle seat

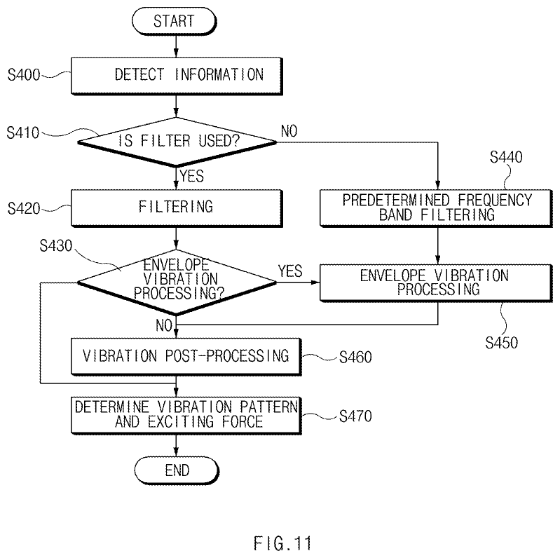

FIG. 11 is a flowchart illustrating a method for determining a vibration pattern and a vibration exciting force according to embodiments of the present disclosure.

A processor 160 of FIG. 1 may process a sound input thereto as a vibration. The processor 160 may receive (or sense) a sound of a sound source (or music) played by a sound output device 140 of FIG. 1.

In S400, the processor 160 may detect environmental information (or vehicle environment information) outside and inside a vehicle using a detection device 120 of FIG. 1. The vehicle environment information may include at least one of pieces of information such as a seat environment, a driving environment, a sound of played music, or a surrounding image (or a surrounding situation).

In S410, the processor 160 may determine whether to use a low pass filter. The processor 160 may determine whether to use a sound of any frequency band in the received sound to implement a vibration.

When it is determined that the low pass filter is used, in S420, the processor 160 may filter a low frequency band. The processor 160 may extract a low-pitched sound from the played music.

In S430, the processor 160 may determine whether to perform envelope vibration processing for the filtered sound. The processor 160 may determine whether to perform envelope vibration processing for the low-pitched sound.

When it is determined that the low pass filter is not used, in S440, the processor 160 may filter a predetermined frequency band (e.g., a high frequency band or a high-pitched portion) in the sound. For example, when the processor 160 wants to implement a voice in music as a vibration, it may determine that the low pass filter is not used. When the low pass filter is not used, the processor 160 may extract a sound of a predetermined specific frequency band from music.

When it is determined that the envelope vibration processing for the low pass filtered sound is performed in S430, in S450, the processor 160 may perform the envelope vibration processing.

When it is determined that the envelope vibration processing for the low pass filtered sound is not performed in S430 or after performing the envelope vibration processing in S450, in S460, the processor 160 may perform vibration post-processing. The envelope vibration processing may be logic for generating a specific frequency as much as the magnitude of the input waveform, which may generate a low-pitched frequency although there is a high-frequency waveform. For example, when only a voice region which is a high frequency is filtered to perform the envelope vibration processing, the same vibration as a voice may occur.

In S470, the processor 160 may proceed with vibration correction using the low pass filtered signal and/or the envelope vibration processed signal to determine a vibration pattern and a vibration exciting force. The processor 160 may implement a seat vibration based on information such as a seat environment, a driving environment, a played music sound, and/or a surrounding image. Thereafter, the processor 160 may control a seat driving device 150 of FIG. 1 based on the determined vibration pattern and the determined vibration exciting force. The seat driving device 150 may generate a seat vibration based on the determined vibration pattern and the determined vibration exciting force under control of the processor 160.

Embodiments of the present disclosure may separate a sound source for each instrument based on passenger emotion when playing music content in a vehicle and may distribute the separated sound source for each instrument to a speaker to play the sound source for each instrument, thus providing a sound of chamber orchestra emotion.

Furthermore, embodiments of the present disclosure may excite a vibration seat based on the sound source for each instrument, which is separated from music content which is being played in the vehicle, with regard to a vehicle environment and driver emotion, further helping the passengers refresh themselves.

Hereinabove, although the present disclosure has been described with reference to exemplary embodiments and the accompanying drawings, the present disclosure is not limited thereto, but may be variously modified and altered by those skilled in the art to which the present disclosure pertains without departing from the spirit and scope of the present disclosure claimed in the following claims. Therefore, embodiments of the present disclosure are not intended to limit the technical spirit of the present disclosure, but provided only for the illustrative purpose. The scope of the present disclosure should be construed on the basis of the accompanying claims, and all the technical ideas within the scope equivalent to the claims should be included in the scope of the present disclosure.

What is claimed is:

1. An emotional care apparatus, comprising:
a sound output device configured to selectively output a sound to a plurality of speakers; and
a processor in communication with the sound output device,
wherein the processor is configured to:
select an emotional care mode;
separate a sound source comprising a plurality of instrument sounds into separated instrument sounds based on the emotional care mode;
distribute the sound source and separated instrument sounds to at least one distributed speaker of the plurality of speakers; and
control the sound output device to output the sound source and separated instrument sounds to the at least one distributed speaker.

2. The emotional care apparatus of claim 1, wherein the processor is further configured to distribute the sound source and separated instrument sounds to the plurality of speakers based on a position of a passenger in a vehicle.

3. The emotional care apparatus of claim 1, wherein the processor is further configured to distribute the sound source and separated instrument sounds to the plurality of speakers based on a frequency for each instrument sound of the plurality of instrument sounds.

4. The emotional care apparatus of claim 1, wherein the processor is further configured to correct a volume level of the sound source and separated instrument sounds depending on the emotional care mode.

5. The emotional care apparatus of claim 1, wherein the processor is further configured to modulate a waveform of the sound source and separated instrument sounds depending on the emotional care mode.

6. The emotional care apparatus of claim 1, wherein the processor is further configured to:
generate an emotional vibration signal based on the sound source for each instrument sound of the plurality of instrument sounds; and
control a vibration seat to be excited based on the emotional vibration signal.

7. The emotional care apparatus of claim 6, wherein the processor is further configured to:
convert the sound source and separated instrument sounds into a converted vibration signal;
synthesize a modulation signal of a main vibration signal and a sub-vibration signal with the converted vibration signal into a synthesized vibration signal; and
correct the synthesized vibration signal to generate the emotional vibration signal.

8. The emotional care apparatus of claim 7, wherein the main vibration signal is a sine wave, and
wherein the sub-vibration signal is a square wave, a triangle wave, or a sawtooth wave.

9. The emotional care apparatus of claim 7, wherein the processor is further configured to:
generate a first vibration at a first point of a seat back based on the main vibration signal; and
generate a second vibration at a second point adjacent the seat back and a seat cushion based on the sub-vibration signal.

10. The emotional care apparatus of claim 1, wherein the processor is further configured to determine the emotional care mode based on at least one of: a user input, a vehicle environment, and/or a passenger emotion state received from a user interface.

11. An emotional care method, comprising:
selecting, by a processor, an emotional care mode;
separating, by the processor, a sound source comprising a plurality of instrument sounds into separated instrument sounds based on the emotional care mode;
distributing, by the processor, the sound source and separated instrument sounds to at least one distributed speaker of a plurality of speakers in a vehicle; and
controlling, by the processor, a sound output device to output the sound source and separated instrument sounds to the at least one distributed speaker.

12. The emotional care method of claim, 11, wherein the distributing of the sound source and separated instrument sounds step further includes:
distributing the sound source and separated instrument sounds to the at least one distributed speaker based on a position of a passenger in the vehicle.

13. The emotional care method of claim 11, wherein the distributing of the sound source and separated instrument sounds step further includes:
distributing the sound source and separated instrument sounds to the plurality of speakers based on a frequency for each instrument sound of the plurality of instrument sounds.

14. The emotional care method of claim 11, wherein the distributing of the sound source and separated instrument sounds step includes:
correcting a volume level of the sound source and separated instrument sounds depending on the emotional care mode.

15. The emotional care method of claim 11, wherein the distributing of the sound source and separated instrument sounds step further includes:
modulating a waveform of the sound source and separated instrument sounds depending on the emotional care mode.

16. The emotional care method of claim 11, further comprising:
generating an emotional vibration signal based on the sound source for each instrument sound of the plurality of instrument sounds; and
controlling a vibration seat to be excited based on the emotional vibration signal.

17. The emotional care method of claim, 16, wherein the generating of the emotional vibration signal step further includes:
converting the sound source and separated instrument sounds into a converted vibration signal;
synthesizing a modulation signal of a main vibration signal and a sub-vibration signal with the converted vibration signal into a synthesized vibration signal; and
correcting the synthesized vibration signal to generate the emotional vibration signal.

18. The emotional care method of claim 17, wherein the main vibration signal is a sine wave, and
wherein the sub-vibration signal is a square wave, a triangle wave, or a sawtooth wave.

19. The emotional care method of claim 17, wherein the controlling of the vibration seat to be excited step further includes:
generating a first vibration at a first point of a seat back based on the main vibration signal; and
generating a second vibration at a second point adjacent the seat back and a seat cushion based on the sub-vibration signal.

20. The emotional care method of claim 11, wherein the selecting of the emotional care mode step further includes:
determining the emotional care mode based on at least one of: a user input, a vehicle environment, and/or a passenger emotion state received from a user interface.
